(19) United States
(12) Patent Application Publication
(10) Pub. No.: US 2024/0108799 A1
(43) Pub. Date: Apr. 4, 2024

(54) PULSATILE FLUSHING OF MEDICAL DEVICES

(71) Applicant: Mokita Medical GmbH, Berlin (DE)

(72) Inventors: Richard Gerard Vecchiotti, Berlin (DE); Ernest G. Schutt, San Diego, CA (US); Joshua Bough, Sutton, Ely (GB); Rosemary Burnell, Cambridge (GB); Tilo Kölbel, Hamburg (DE); Andrew Macleod, Harston (GB)

(73) Assignee: Mokita Medical GmbH, Berlin (DE)

(21) Appl. No.: 18/257,613

(22) PCT Filed: Dec. 15, 2021
(86) PCT No.: PCT/EP2021/085936

§ 371 Date: Jun. 15, 2023

Related U.S. Application Data
(60) Provisional application No. 63/126,351, filed on Dec. 16, 2020.

(30) Foreign Application Priority Data
Jan. 28, 2021 (GB) 2101194.5

(51) Int. Cl. A61M 5/14

(52) U.S. Cl. CPC A61M 5/14; A61M 2005/1402; A61M 2005/1403; A61M 2205/3334

(57) ABSTRACT

A system for flushing a lumen of a medical device to remove air, comprising: a first fluid delivery device adapted to provide a pulsatile flow of a flushing gas from a pressurised source of the flushing gas; a second fluid delivery device adapted to provide a pulsatile flow of a flushing liquid from a source of the flushing liquid; and, at least one fluid coupling for connecting the first fluid delivery device and the second fluid delivery device to the lumen of the medical device.

### BACKGROUND

Procedures that intervene with vasculature that is in communication with the cerebral vasculature can put patients at risk of cerebral injury if gaseous volumes enter the blood stream.

It is standard practice to flushing medical devices with a flushing fluid, such as medical grade saline, prior to an intravenous procedure to displace air from medical devices in order to reduce the risk of air entering the blood stream.

In EP 3 367 978 A1, it was proposed to use a multi-stage flushing method to flush a stent-graft in order to remove air from the stent-graft prior to it being inserted into a patient. The first stage involves flushing the stent-graft with a fluid such as carbon dioxide to displace air from the stent-graft, and the second stage involves flushing the stent-graft with a solution that preferentially absorbs air, such as a perfluorocarbon solution or a degassed solution.

While such methods result in improved removal of air from medical devices, the danger posed even by minuscule volumes of air means that there remains a need for improved air removal methods and systems.

### SUMMARY OF THE INVENTION

According to a first aspect of the invention, there is provided a system for flushing a lumen of a medical device to remove air, comprising: a first fluid delivery device adapted to provide a pulsatile flow of a flushing gas from a pressurised source of the flushing gas; a second fluid delivery device adapted to provide a pulsatile flow of a flushing liquid from a source of the flushing liquid; and, at least one fluid coupling for connecting the first fluid delivery device and the second fluid delivery device to the lumen of the medical device.

The inventors have recognised that flushing a lumen of a medical device using a pulsed/pulsatile flow of a flushing fluid increases the efficacy of air removal during the flushing process. In conventional flushing techniques, the flow of the flushing fluid is laminar, meaning that the fluid flow rate is slower closer to internal-surface-walls of a lumen in which the fluid is flowing. At each surface, an infinitesimal layer of the flushing fluid does not flow (i.e. it has zero velocity), which reduces the effectiveness of flushing. This is especially problematic when air bubbles remain and collect on internal-surface-walls of delivery system lumens, on external surfaces of implants contained within delivery system lumens, and in-between external surfaces of implants to be delivered and internal walls of delivery systems lumens-, as these bubbles can be difficult to dislodge when flushing using laminar flow.

Pulsed flow disrupts the development of laminar flow and brings the velocity profile closer to that of turbulent or plug flow. The higher degree to which this effect occurs further increases the speed of the fluid close to surfaces. Pulsed flow therefore increases the flushing effectiveness in regions where flushing is most challenging.

As described herein, the term air should be understood to refer to the invisible gaseous substance surrounding the earth, which is a mixture mainly of oxygen and nitrogen.

Preferably, the first fluid delivery device comprises a flow restrictor that is actuatable between an open position (in which the flushing gas can flow) and a closed position (in which the flushing gas cannot flow) to thereby pulse a flow of the flushing gas.

Optionally, the first fluid delivery device may comprise a compression element configured to compress a flexible tube coupled to the source of the flushing gas at predetermined time intervals to thereby restrict a flow of the flushing gas through the tube. This arrangement allows the fluid delivery device to use off-the-shelf connecting tubes and sources of flushing gas.

The compression element may reciprocally movable, for example by a solenoid motor.

Alternatively, the compression element may be a rotatable cam.

Alternatively, the compression element may be pneumatically drivable. For example, the compression element may be configured to operate as a pneumatic reciprocation circuit. The flushing gas may optionally serve as a source of pneumatic power.

The rotatable cam may be coupled to a torsion spring adapted to drive the rotatable cam (i.e. a clockwork mechanism). This means the first fluid delivery device does not require a power source, making it extremely versatile. Alternatively, the compression element may be coupled to an electric motor configured to drive the compression element. The electric motor may be battery or mains driven, for example.

In some examples, the first fluid delivery device may comprise a user actuatable (i.e. manually operated) gas control mechanism for providing the pulsatile flow of the flushing gas. For example, first fluid delivery device may comprise a user actuatable trigger configured to pulse the flushing gas upon application of an actuating force (e.g. applied by the user/operator)

The system may further comprise a stand adapted to retain a source of the flushing gas in an upright position. The flushing source may be a gas such as carbon dioxide (CO2). If a CO2 source (e.g. cannister) is not held upright, then liquid CO2 may be expelled from the source, which leads to faster depletion of the CO2.

The stand may optionally comprise the first fluid delivery device. That is, the first fluid delivery device and the stand may be formed as a single component.

The stand may optionally comprise both the first fluid delivery device and the second fluid delivery device. That is, the first fluid delivery device, the second fluid delivery device, and the stand may be formed as a single component.

Preferably, the second fluid delivery device comprises a user actuatable liquid delivery mechanism, such as a trigger, for providing the pulsatile flow of the flushing liquid.

In addition, the second fluid delivery device may comprise a user actuatable positive displacement pump, which may comprise a compression chamber.

The positive displacement pump may further comprise a one-way valve configured to allow a one-way flow of flushing liquid from the source of the flushing liquid into the compression chamber (i.e. when the trigger is actuated). For example, the one-way valve may be an umbrella valve or a duckbill valve or similar.

Preferably, the positive displacement pump also comprises a one-way valve configured to allow a one-way flow of flushing liquid from the positive displacement pump towards the at least one fluid coupling. The one-way valve may again be an umbrella valve or a duckbill valve or similar.

The positive displacement pump may comprise one or more resilient compression members arranged to reset the user actuatable liquid delivery mechanism to an initial position and/or draw the flushing liquid from the source of flushing liquid fluid source.

Optionally, the fluid coupling may be a three-way valve.

In some examples, the first and second fluid delivery devices may be comprised in a single device.

Alternatively, the first fluid delivery device may be couplable (or coupled) to the fluid coupling via the second fluid delivery device.

The flushing liquid may be a first flushing liquid, and the second fluid delivery device may be further adapted to provide a pulsatile flow of a second flushing liquid from a source of the second flushing liquid.

Preferably, the second fluid delivery device further comprises control means for selectively coupling the second fluid delivery device to the respective sources of the first and the second flushing liquids.

The system may additionally comprise a sterile filter arranged or positionable in-line between the first fluid delivery device and the pressurised source of the flushing gas.

According to a second aspect of the invention, there is provided a method for flushing a lumen of a medical device to remove air prior to introducing the medical device within a body, comprising: flushing the lumen with a pulsed supply of a flushing fluid.

As explained earlier, the inventors have recognised that flushing a lumen of a medical device using a pulsed/pulsatile flow of a flushing fluid increases the efficacy of air removal during the flushing process. In particular, the pulsatile flow improves the displacement of air by creating a turbulent flow of flushing fluid that disrupts laminar flow and increases the speed of the flushing fluid close to surfaces, thereby increasing flushing effectiveness in regions where flushing is most challenging.

Multiple flushing fluids may be used to sequentially flush the lumen, and multiple lumens may be flushed simultaneously.

While it is preferable that all flushing fluids are pulsed when using multiple flushing fluids in sequence, this is not essential for improving the efficacy of the flushing. When flushing with multiple flushing fluids, only one of these (preferably the first flushing fluid used, e.g. carbon dioxide) needs to be pulsed in order to provide the effect of improved air removal during flushing. However, pulsing more than one of the flushing fluids may further enhance this effect.

Optionally, the flushing fluid may be a flushing gas. For example, the flushing gas may be carbon dioxide. Preferably, the method further comprises flushing the lumen with a pulsed supply of a flushing liquid.

Alternatively, the flushing fluid may be a flushing liquid. For example, the flushing liquid may be saline or perfluorocarbon. The flushing liquid may be a buffer solution, pH adjusted and or degassed. The flushing liquid may be a gas absorbing liquid (e.g. a degassed and/or pH adjusted solution that absorbs air and/or carbon dioxide).

Multiple flushing liquids may be used. For example, a first flushing liquid (such as a gas absorbing liquid) may be used followed by flushing with saline. Either or both of these liquid flushes may be pulsed.

Flushing the lumen may comprise flushing at a pressure above 101.325 kPa (also known as standard pressure, which is approximately equal to the air pressure at the Earth's surface). Flushing at increased pressure improves the flushing fluid's ability to absorb air.

Preferably, flushing the lumen comprises coupling the lumen to the pulsed supply of flushing fluid.

According to a third aspect of the invention, there is provided a bubble capture device for capturing gas bubbles entrained in a flow of a liquid, comprising: a vent port for venting gas bubbles from the bubble capture device; an inlet port for receiving the flow of the liquid; an outlet port; and, a fluid conduit coupling the inlet port to the outlet port, wherein the fluid conduit comprises at least one baffle arranged to (in use) modify or agitate the flow of the liquid (i.e. flowing through the fluid coupling) to thereby separate gas bubbles from the flow of the liquid.

The bubble capture device may also be referred to as a priming module.

The bubble capture device creates a tortuous flow path for the flushing liquid, which causes gas bubbles entrained in the flow of flushing liquid to separate out such that they can be vented via the vent port. When used in combination with a flushing system such as the first aspect, this helps to ensure that all air is removed from the flushing system itself during the priming process, rather than allowing this air to reach the medical device. The bubble capture device therefore allows for enhanced priming of flushing systems by improving the removal of air during the priming process.

Preferably, the bubble capture device is shaped to direct or channel separated gas bubbles towards the vent port. For example, at least one surface may be angled towards the vent port when in use. That is, at least one surface (preferably a top surface) of the bubble capture device may be slanted/sloped/inclined towards the vent port, with the vent port positioned at a topmost point/apex of the bubble capture device.

The baffles may also be referred to as deflectors, flow deflectors, flow disruptors, agitators, agitating elements, agitating components or similar, and may be any component that disrupts, agitates or redirects the flow of fluid. The baffles may be ribs, separate components or moulded components for example.

The purpose of the baffles is to create a tortuous flow path through the priming module for the flushing liquid (i.e. the baffles are arranged to create a tortuous flow path through the fluid conduit). This results in the flushing fluid being agitated by the baffles, thereby separating gas bubbles (such as air) from the flow of flushing liquid.

The at least one baffle may formed of an elastomeric material, such as silicone, or a rigid material, such as polycarbonate. Air bubbles preferentially coalesce on elastomeric materials, so using an elastomeric material for the buffer enhances the separation of gas from the flushing liquid.

The at least one baffle may be arranged to interrupt laminar flow of the flow of the liquid within the conduit.

Preferably, the at least one baffle comprises one or more angled projections, such as angled edges or corners. The relatively sharp edges presented by these angled projections further agitate the flow of liquid, thereby enhancing gas separation.

Optionally, the inlet port and the outlet port may be arranged on opposing sides of the bubble capture device, and the at least one baffle may be positioned between the inlet port and the outlet port and arranged perpendicular to a line intersecting the inlet port and the outlet port (i.e. perpendicular to an alignment axis of the inlet port and outlet port).

The vent port may optionally be sealable, for example by a vent cap or similar.

The vent port may also be shaped to couple to a syringe, such as a vacuum pressure syringe.

Optionally, an outer casing of the bubble capture device may be formed of an elastomeric material such as silicone. Alternatively, the outer case may be formed of a rigid material such as polycarbonate.

According to another aspect of the invention, there is provided a catheter flushing system comprising: a source of flushing liquid; a pump for driving the flushing liquid; and, the bubble capture device of the third aspect, wherein the inlet port of the bubble capture device is couplable (or coupled) to the source of flushing liquid.

The pump may also be couplable (or coupled) to the inlet port of the bubble capture device. That is, the source of flushing liquid and the pump may both be couplable to the inlet port such that the pump is arranged to drive the flushing liquid through the inlet port.

Alternatively, the pump may be couplable (or coupled) to the vent port of the bubble capture device. That is, the pump may be arranged to draw/aspirate/suck the flushing liquid into the bubble capture device through the inlet port by applying a negative pressure at the vent port.

The pump may be any suitable pumping device, such as a syringe (e.g. vacuum pressure syringe) or similar.

Using a bubble capture device in combination with a catheter flushing system allows for improved priming of the catheter flushing system by enhancing air removal during priming.

According to yet another aspect of the invention, there is provided a method of priming a catheter flushing system, comprising: coupling a source of flushing liquid to an input port of a bubble capture device; opening a vent on the bubble capture device; driving a flow of the flushing liquid through the bubble capture device and out of the vent port; and, closing the vent port.

Using a bubble capture device in combination with a catheter flushing system results in improved priming of the catheter flushing system by enhancing air removal during priming.

The flow of the flushing liquid may be driven by a pump coupled to the source of the flushing liquid. Alternatively, the flow of the flushing fluid may be driven by a vacuum source coupled to the vent port of the bubble capture device.

### BRIEF DESCRIPTION OF THE DRAWINGS

Examples of the present invention will now be described in detail with reference to the accompanying drawings, in which:

FIGS. 1-7 show systems for flushing medical devices with pulsatile flows of flushing fluids;

FIG. 8 shows a fluid delivery device for use in the systems of FIGS. 2, 4, 5 and 6;

FIG. 9 shows a cross-sectional view of the fluid delivery device of FIG. 8;

FIGS. 10a-10d show rear views of the fluid delivery device of FIG. 8;

FIG. 11a shows an alternative fluid delivery device;

FIG. 11b shows a close-up view of a trigger of the fluid delivery device of FIG. 11a;

FIGS. 12a and 12b show gas sources for use in the systems of FIGS. 1-7;

FIG. 13 shows a fluid delivery device for generating a pulsatile flow of a flushing fluid;

FIG. 14 shows exemplary packaging for supplying parts of a flushing system;

FIG. 15 shows a bubble capture device;

FIGS. 16a-c show alternative bubble capture devices;

FIGS. 17a-c show another alternative bubble capture device;

FIG. 18 shows an alternative fluid delivery device for generating a pulsative flow of a flushing gas;

FIG. 19 shows a system for flushing medical devices incorporating the bubble capture device and alternative fluid delivery device;

FIGS. 20a and 20b show another alternative fluid delivery device for generating a pulsatile flow of a flushing gas;

FIG. 21 illustrates a method for flushing a medical device, and,

FIG. 22 illustrates a method for priming a flushing system.

### DETAILED DESCRIPTION

The present disclosure relates to systems, devices and methods for use when flushing medical devices to remove air. Medical devices, such as catheters and stents, used in endovascular and percutaneous procedures are generally packaged in sterile environments to mitigate the risk of infection caused by microbiological contamination.

However, these medical devices must still be flushed prior to use in order to remove air and other impurities. There is a growing recognition that residual volumes of air released during endovascular and percutaneous procedures can result in cerebral injury. Complete removal of air from medical devices remains a challenge, especially for devices such as stents that have numerous passages and crevices that can trap small volumes of air in way that makes them difficult to remove using conventional flushing methods.

The present inventors have recognised that the efficacy of air removal during flushing can be improved by using a pulsed/pulsatile flow of a flushing fluid. Pulsed flow enhances the flushing process by disrupting the development laminar flow and bringing the velocity profile of the flushing fluid closer to that of turbulent or plug flow. The higher degree to which effect occurs further increases the speed of the fluid close to surfaces. Pulsed flow therefore increases the flushing effectiveness in regions where flushing is most challenging.

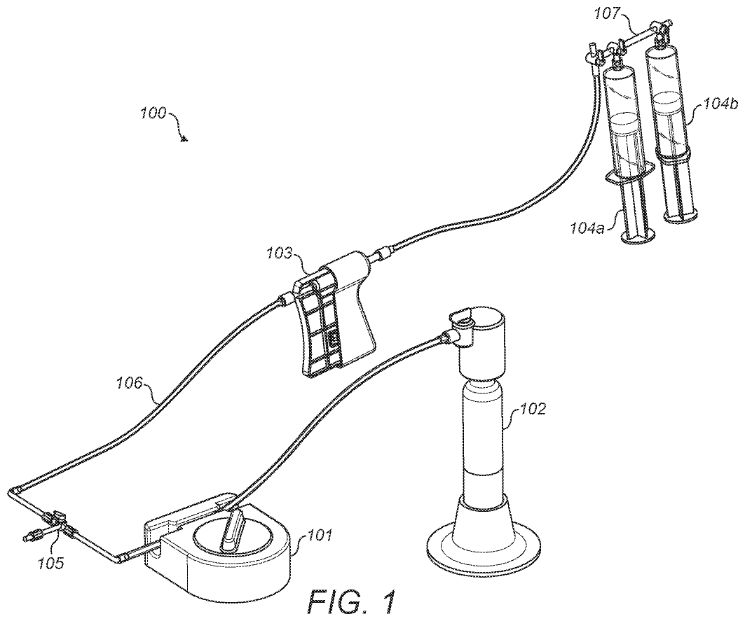

FIG. 1 shows a system 100 for flushing medical devices with a pulsatile flow. The system comprises a pulse generator 101 coupled to a flushing gas source 102 and a fluid coupling 105. The system also features a syringe pump 103 coupled to the fluid coupling 105 and to two fluid syringes 104a, 104b via a syringe manifold 107. All components of the system are coupled via connecting tubes 106, which may use any suitable connections (such as push fit, interference fit, luer locks etc.).

The pulse generator 101, which is a first fluid delivery device of the system 100, is adapted to provide a pulsatile flow of flushing gas from the flushing gas source 102 to the fluid coupling 105. The illustrated fluid coupling 105 is a three-way valve, which may be controlled to selectively couple the flushing gas or flushing fluids from the fluid syringes 104a, 104b to a medical device (not shown). The fluid coupling 105 may alternatively be referred to as a valve manifold and may take alternative forms (such as being a simple three-way connector with no valve). Alternatively, the fluid coupling 105 may have only two connections or be a connector at an end of a connecting tube.

As discussed in more detail later, the pulse generator 101 may be electric or mechanical, and is arranged to pulse the supply of gas from the flushing gas source 102 at regular intervals (e.g. by compressing the connecting tube 106 between the flushing gas source 102 and the fluid coupling 105 at regular intervals to thereby restrict/interrupt the flow of the flushing gas).

The syringe pump 103, which is a second fluid delivery device of the system 100, is a user actuatable pump arranged to draw flushing fluids (in particular, flushing liquids) from the fluid syringes 104a, 104b and deliver a flow of flushing fluid to the medical device via the fluid coupling 105. The number of fluid syringes 104a, 104b will depend upon the number of flushing fluids being used to flush the medical device. In the illustrated system 100 there are two fluid syringes 104a, 104b, but additional or fewer syringes could be used as required. The syringe manifold 107 has one or more valves positioned to selectively couple each of the fluid syringes 104a, 104b to the syringe pump 103 as required. It should be understood that the syringe pump 103 could be any pumping means capable of pumping an uncompressed source of fluid from a fluid source to the medical device. Similarly, the fluid coupling 105 and syringe manifold 107 could be any manifold suitable for selectively coupling fluid containers and connecting tubes and may use any suitable connection interface.

Pulsed flow of flushing fluid from the fluid syringes 104a, 104b can be achieved by the user manually actuating the syringe pump 103 at regular intervals, as described in more detail later.

In some examples, the flushing gas source 102 may be a canister or similar containing compressed/pressurised carbon dioxide (CO2). The fluid syringes 104a, 104b may contain a sterile liquid such as perfluorocarbon solution or saline, which may optionally be a buffer solution, degassed and/or pH adjusted.

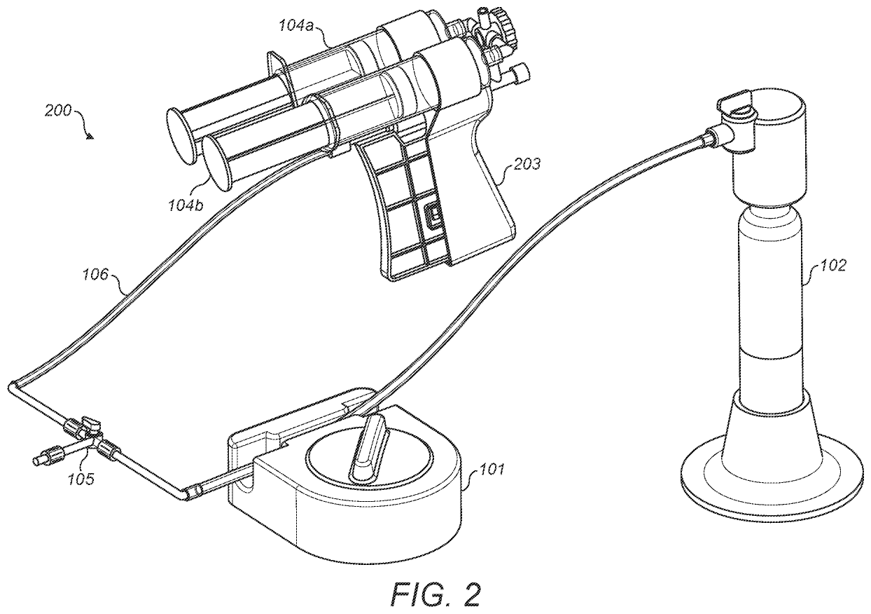

An alternative system 200 is shown in FIG. 2. The system 200 features the same pulse generator 101 and source of flushing gas 102, but the syringe pump 103 of the previous system 100 is replaced by a syringe pump 203 in which the fluid syringes 104a, 104b are housed in the syringe pump 203 (i.e. not connected via connecting tubes). The fluid syringes 104a, 104b are preferably removable from the syringe pump 203 to allow the fluid sources to be changed and replaced as necessary. As will be described in more detail later, the syringe pump 203 preferably features user actuatable control means for selecting between the fluid syringes 104a, 104b. The system 200 is otherwise operated in the same manner as the earlier system 100.

Compared to the system 100 of FIG. 1, the system 200 of FIG. 2 is more compact and requires fewer connecting tubes, which also reduces the likelihood of a connection being broken during the flushing procedure. However, the syringe pump 103 of the system 100 in FIG. 1 is slightly simpler and potentially cheaper to manufacture that the syringe pump 203 shown in FIG. 2.

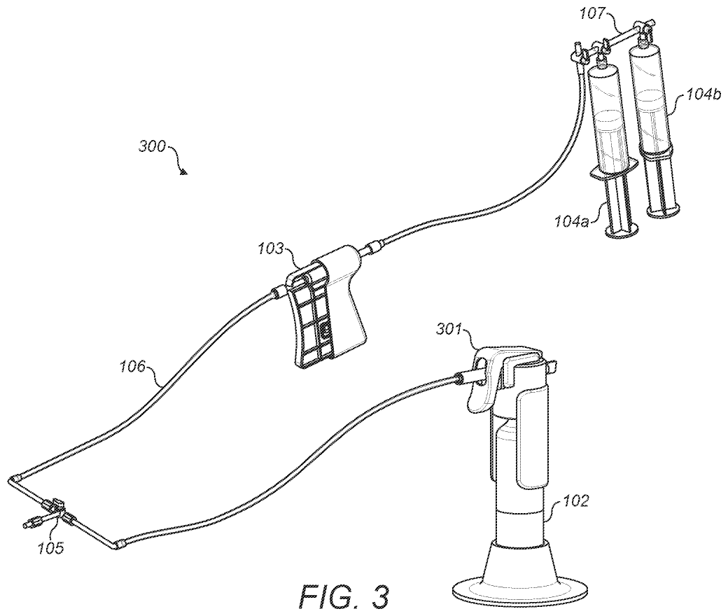

Another alternative system 300 is shown in FIG. 3. The syringe pump 103 and fluid syringes 104a, 104b as the same as of the system 100 shown in FIG. 1, but the pulse generator has been replaced by a user actuatable (i.e. manually-operated) gas trigger 301. Rather than using an electric or mechanical pulse generator to provide regular pulses, the user instead squeezes the gas trigger 301 at regular intervals to interrupt the flow of flushing gas from the source of flushing gas 102. It should be noted that the gas trigger 301 could also be adapted to provide a flow of gas when the trigger is squeezed (instead of interrupting the flow of gas) and pulses could be generated in an analogous manner.

While the illustrated gas trigger 301 is shown in a preferable configuration in which it is directly connected to a cannister of flushing gas, alternatives are envisaged in which an in-line trigger could be used (i.e. coupled to the flushing gas source 102 via one or more connecting tubes).

Compared to the systems 100 and 200 in FIGS. 1 and 2, the system 300 in FIG. 3 does not require an electric or mechanical pulse generation means and is therefore simpler and cheaper to manufacture. However, it is slightly more complicated to use because the operator must manually control each pulse of flushing gas.

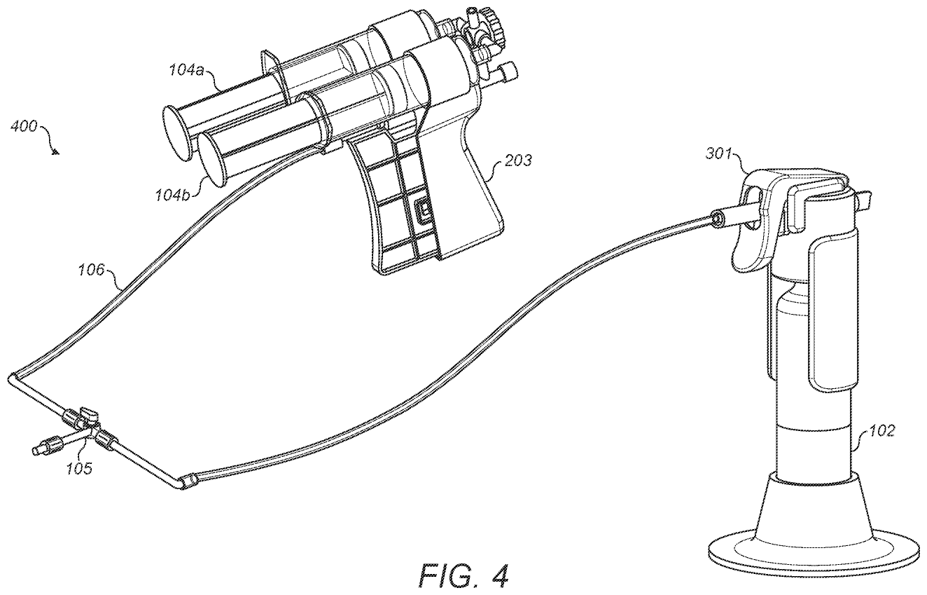

The system 400 illustrated in FIG. 4 effectively combines the syringe pump 203 of FIG. 2 with the gas tigger 301 of FIG. 3.

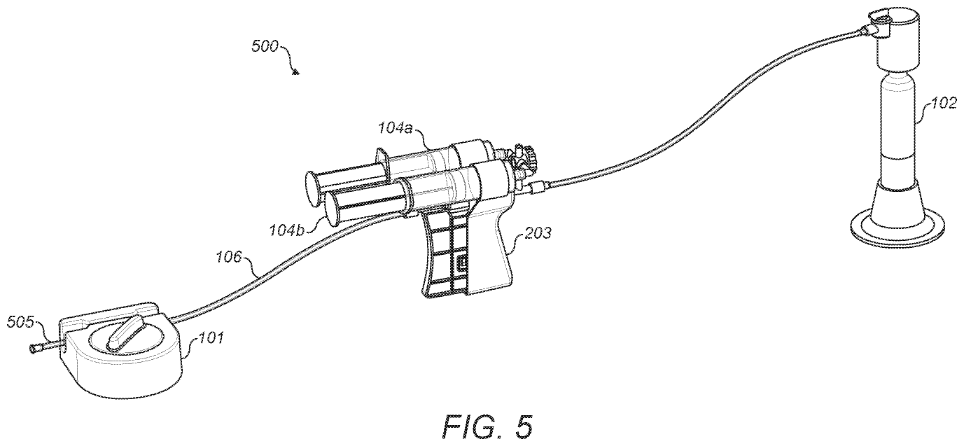

In the systems of each of FIGS. 1-4, the flows of flushing fluids from the flushing gas source 101 and the fluid syringes 104a, 104b are effectively connected to the fluid coupling 105 in parallel. FIGS. 5-7 show examples of alternative systems in which the flushing gas source 102 and fluid syringes 104a, 104b are effectively connected in series.

In each of FIGS. 5-7, the three-way valve used as a fluid coupling 105 in FIG. 104 is replaced with fluid coupling 505 which is a simple connector such as a luer-lock.

In FIG. 5, the components are effectively the same as those in FIG. 2 except that the source of flushing gas 102 is coupled to fluid coupling 505 via the syringe pump 203, and the syringe pump 203 is coupled to the fluid coupling 505 via the pulse generator 101. In this example, pressurised gas from the flushing gas source 102 flows through the syringe pump 203 (i.e. the syringe pump 203 is not used to drive the flow of flushing gas from the flushing gas source 102) and the pulse generator 101 provides the pulsed flow of the flushing gas (e.g. by compressing the connecting tube 106 as described earlier).

When the system 500 is used to deliver a pulsatile flow of flushing fluid from the fluid syringes 104a, 104b, the flow of flushing gas from the flushing gas source 102 is stopped (for example using a valve on the flushing gas source 102) and the syringe pump 203 is then used to pump the flushing fluid from the fluid syringes 104a, 104b as in the earlier embodiments. The pulse generator 101 should be in a passive state at this stage, thereby allowing the unimpeded flow of flushing fluid from driven by the syringe pump 203.

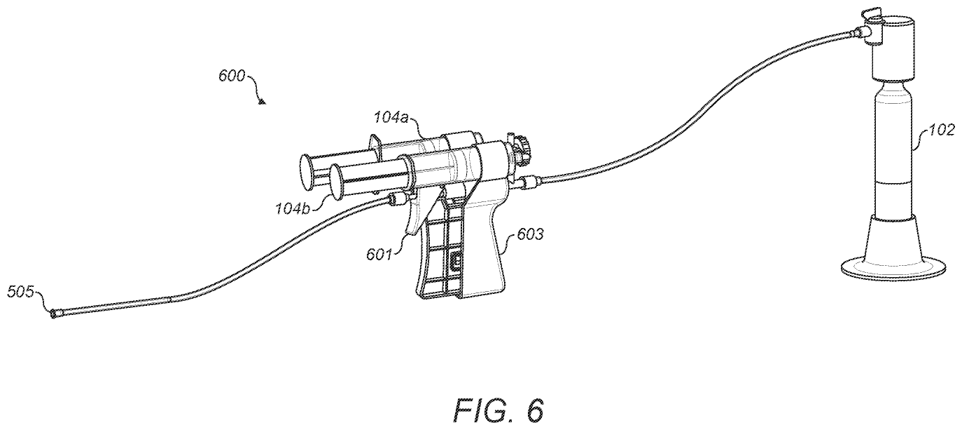

The alternative system 600 shown in FIG. 6 is essentially a hybrid of those shown in FIGS. 3 and 5, except that the gas trigger 301 of FIG. 3 is instead an integral gas trigger 601 integrated into the syringe pump 603. As in FIG. 3, there is no separate electronic or mechanical pulse generator. It should be noted that the gas trigger 301 of FIG. 3 could also be used in the system 600 of FIG. 6 in place of the integral gas trigger 601.

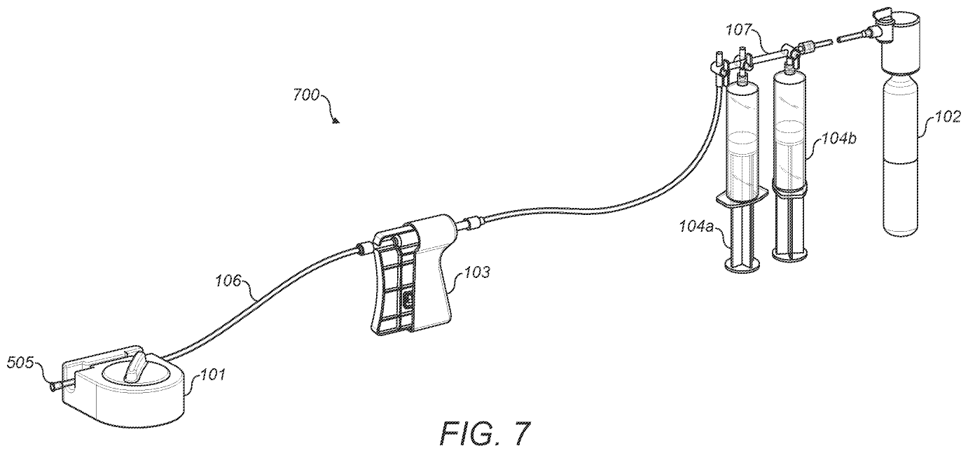

Likewise, the alternative system 700 shown in FIG. 7 is essentially a hybrid of those shown in FIGS. 1 and 5, with the flushing gas source 102 connected to the syringe manifold 107. The valves of the syringe manifold 107 are used to selectively switch between the fluid syringes 104a, 104b and the flushing gas source 102.

One skilled in the art will appreciate that additional modifications can be made to the systems disclosed FIGS. 1-7 by combining different aspects of these systems, and such modifications are envisaged by the inventors. In addition, various components could be integrated together (for example the pulse generator could be integrated into a stand for the flushing gas source).

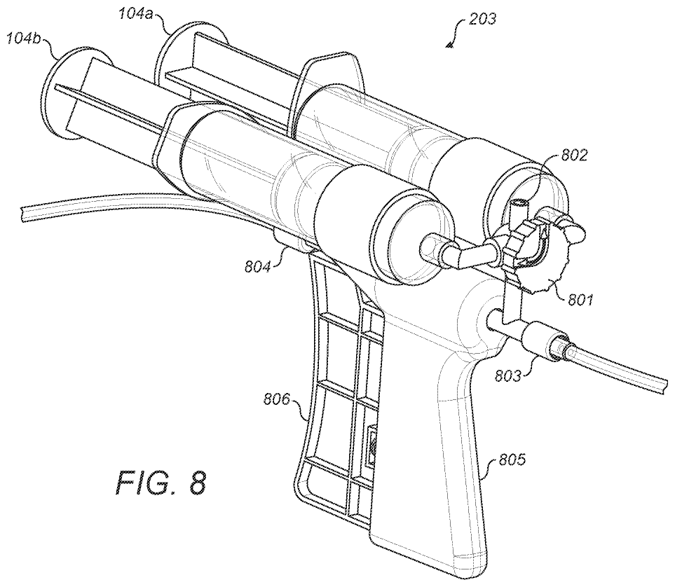

As discussed earlier the syringe pump 203 utilised in the systems of FIGS. 2 and 4-6 has user actuatable control means for selecting between the fluid syringes 104a, 104b. As shown in FIG. 8, which shows a rear view of the syringe pump 203, this user actuatable control means may take the form of a control knob 801 which can be rotated between different positions to thereby selectively couple the syringes 104a, 104b to the pump itself and to a vent port 802.

The syringe pump 203 also features a fluid entry port 803 for coupling to the flushing gas source 102 and a fluid exit port 804 for coupling to the fluid coupling 105, 505.

The syringe pump 203 has a main body 805 shaped to accommodate a trigger component 806. In use, the user holds the syringe pump 203 with the main body 805 in their palm and generates a pulse of flushing fluid from the fluid syringes 104a, 104b by applying an actuating force to press the tigger component 806 into the handle section 805.

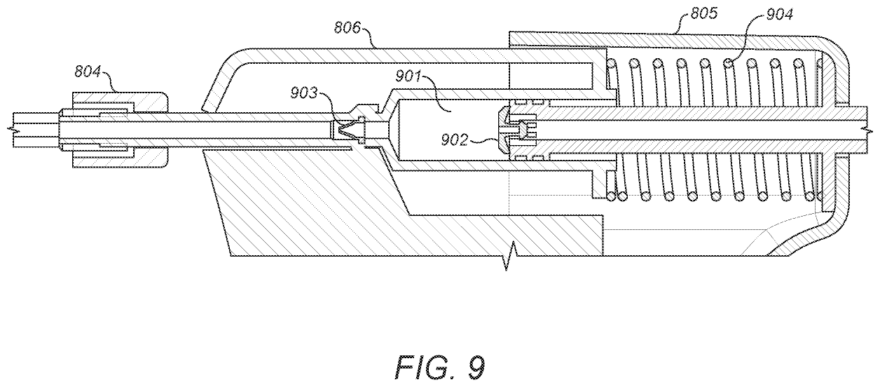

The syringe pump 203 may drive fluid by means of a positive displacement mechanism, as shown in FIG. 9. Positive displacement pumps deliver a fixed volume of fluid each time the pump is actuated.

When the trigger component 806 is actuated, this causes a compression chamber 901 within the syringe pump 203 to be compressed. Any fluid inside the compression chamber 901 will therefore simultaneously experience a compressing force. In the case of a liquid, which is essentially incompressible, this action will cause the liquid to be driven through the duckbill valve 903 located at an outlet of the compression chamber 901. The duckbill valve 903 allows a one-way flow of fluid out of the compression chamber 901, thereby preventing fluid flowing back into the compression chamber 901 through the outlet when the compression chamber 901 expands.

When the trigger component 806 is released, a resilient biasing means, such as the illustrated spring 904, acts to urge/return the trigger component 806 back to its initial position. This action causes the compression chamber 901 to expand to its original size, thereby lowering the pressure inside the compression chamber 901. This causes a volume of liquid to be drawn into the compression chamber 901 via an umbrella valve 902 located at an inlet of the compression chamber 901—this liquid replaces the liquid that was expelled when the compression chamber 901 was compressed. The umbrella valve 902 prevents liquid inside the compression chamber 901 flowing back through the inlet when the compression chamber 901 is compressed.

It should be noted that although the positive displacement mechanism of the syringe pump 203 works more effectively with liquids (as they cannot be compressed very much), the mechanism can also be used to drive gases to some extent (although the compressibility of the gas reduces the effectiveness and means that some of the gas in the compression chamber 901 may be compressed rather than expelled when the compression chamber 901 is compressed).

It should also be noted that the duckbill valve 903 and umbrella valve 902 could be replaced with other one-way valves. For example, both valves could be duckbill/umbrella valves, or one or both may be a different type of valve capable of allowing the flow of fluid in one direction and restricting/preventing the flow of fluid in an opposing direction.

One skilled in the art will appreciate that other arrangements of positive displacement pump are possible and that other pumping mechanisms could also be employed to pump flushing fluids (especially liquids) from the fluid syringes 104a, 104b to the fluid coupling 105, 505.

It will also be understood that pressurised fluid will be able to flow through the positive displacement mechanism provided the pressure is sufficient to overcome the umbrella valve 902 and duckbill valve 902. In this way, gas from the flushing gas source 101 can flow through the positive displacement mechanism relatively unimpeded in the systems illustrated in FIGS. 5-7.

The same positive displacement mechanism may be used by both types of syringe pump 103, 203 shown in FIGS. 1-7.

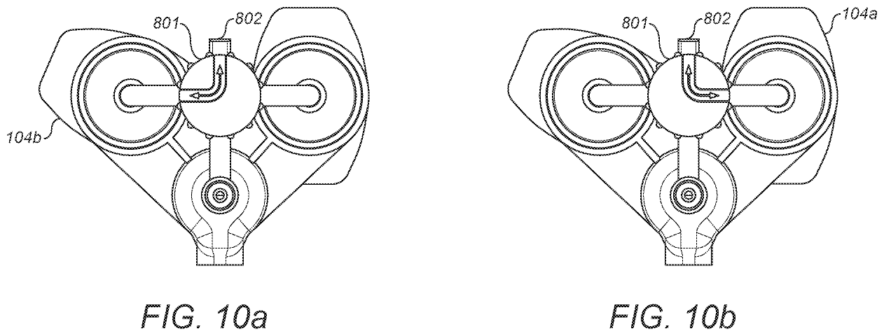

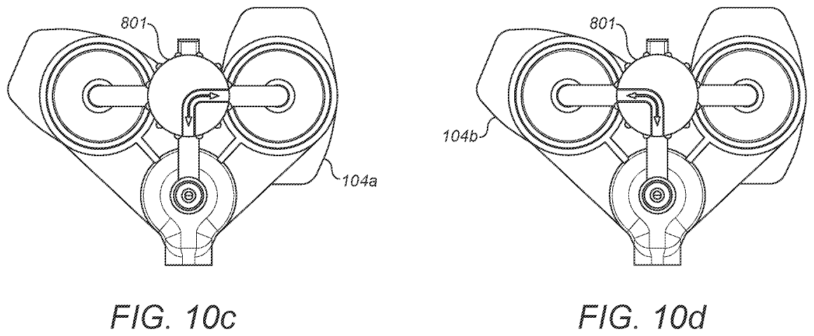

The mechanism of the control knob 801 of the syringe pump 203 is shown in more detail in FIGS. 10-10d. The control knob 801 works by selectively coupling two adjacent fluid paths using a “L-shaped” channel and closing all other fluid paths. In this way, at any one time one (and only one) of the syringes is coupled either to the pumping mechanism (i.e. to the fluid conduit running through the pump via the positive displacement mechanism) or to the vent port 802, and the other is closed.

In FIG. 10a, the control knob 801 is positioned such that the fluid syringe 104b is couped to the vent port 802. In FIG. 10b, the control knob 801 is positioned such that the fluid syringe 104a is couped to the vent port 802. In FIG. 10c, the control knob 801 is positioned such that the fluid syringe 104a is couped to the pumping mechanism. In FIG. 10d, the control knob 801 is positioned such that the fluid syringe 104b is couped to the pumping mechanism.

One skilled in the art will recognise that the above selective coupling mechanism is just one example and that alternative valve configurations and devices may be used to achieve the same result of selectively coupling fluid syringes 104a, 104b to the pump mechanism and/or vent port 802.

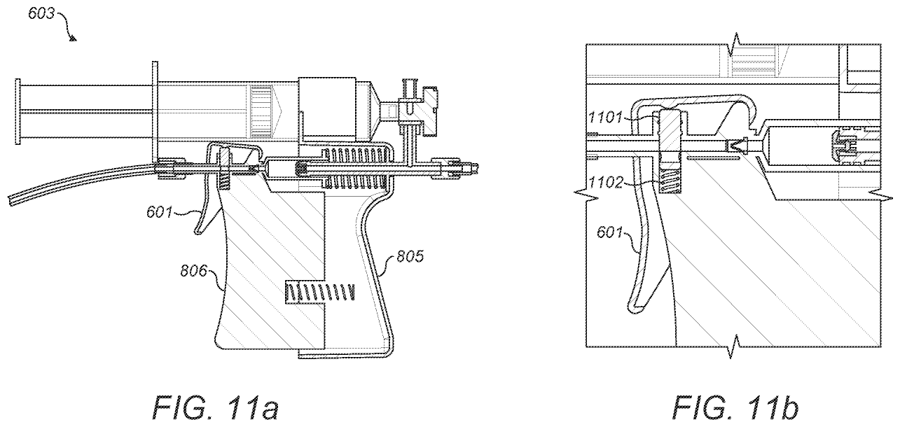

FIGS. 11a and 11b show the syringe pump 603 with the integrated gas trigger 601. As visible in FIG. 11b, which shows the gas trigger 601 in an actuated position, the gas trigger mechanism comprises a valve element 1101 biased by a biasing member such as a spring 1102. When no actuating force is applied to the gas trigger 601, a fluid passage in the valve element 1101 aligns with a fluid passage through the syringe pump 603. When an actuating force is applied to the gas trigger 601, the valve element 1011 is displaced such that the fluid passage in the valve element 1101 is moved out of alignment with the fluid passage through the syringe pump, thereby restricting or preventing the flow of fluid. The spring 1102 acts to return the valve element 1101 and the gas trigger to their initial positions. One skilled in the art will appreciate that this mechanism could be reversed to instead restrict the flow of fluid when the trigger is not actuated and allow the flow of fluid when the trigger is actuated.

A similar mechanism can be used for the gas trigger 301 shown in FIGS. 3 and 4.

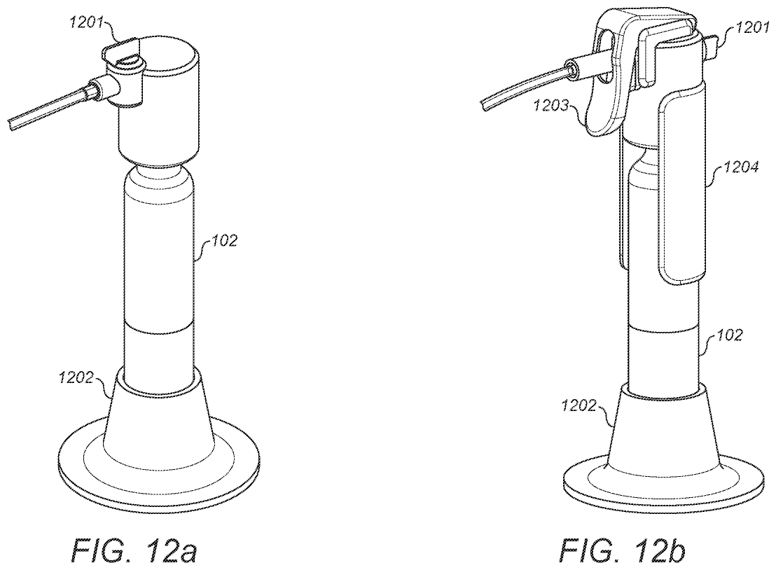

Moving on to FIGS. 12a and 12b, two examples of the flushing gas source 102 are shown. FIG. 12a shows the arrangement used in the systems of FIGS. 1, 2, 5 and 6 (and optionally 7), and FIG. 12b shows the arrangement used in the systems of FIGS. 3 and 4.

In both figures, the flushing gas source 102 is a cannister of pressurised or compressed gas retained in an upright position by a stand 1202. It is important that the cannister is retained in an upright position when using certain gases such as CO2, because otherwise liquid CO2 could be expelled from the cannister instead of CO2 gas, which in turn will lead to the CO2 being depleted much faster. Although not illustrated in the figures, a sterile filter component/disc is preferably positioned in line it the flushing gas source 102.

In both figures, the flow of flushing gas is controllable by a valve 1201.

In FIG. 12b, the flushing gas source 102 is provided with a gas trigger 301 which works using a similar principle to that shown in FIG. 11b, as discussed earlier. In addition, because the user may have to grip the flushing gas source 102 to actuate the trigger, the cannister is provided with a shield element 1204 to reduce heat conduction between the user's hand and the cannister (the cannister may become extremely cold due to the expansion of gases occurring inside the cannister during use).

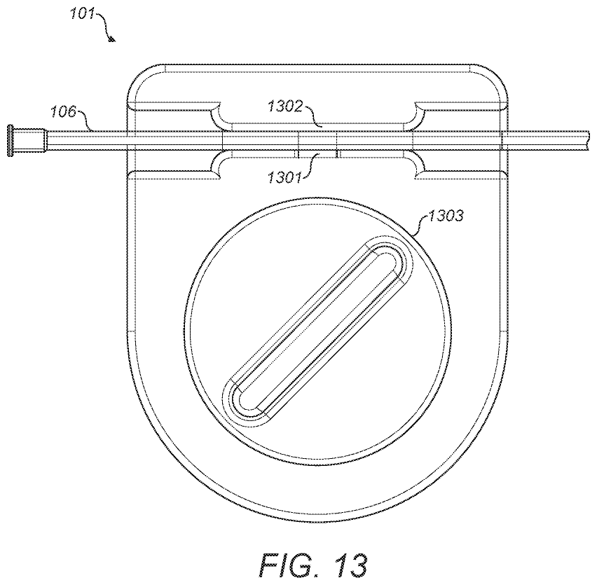

The pulse generator 101 of some of the earlier systems is shown in FIG. 13. The pulse generator 101 comprises a compression element 1301 configured to compress a section of connecting tube 106 retained in a groove or channel 1302 of the pulse generator 101. The illustrated compression element 1301 is configured to move reciprocally when in use to thereby compress and release the connecting tube 106. The pulse generator 101 may be referred to as being in a closed position when it is preventing the flow of flushing gas and in an open position when it is permitting the flow of flushing gas.

Alternative compression elements are envisaged in which the compression element is a rotating cam element that compresses and released the connecting tube 106 as it rotates. The exact nature of the compression element 1301 and the channel 1302 is not essential for operation of the pulse generator: what is essential is that the pulse generator is operable to in some way restrict the flow of fluid passing through it at regular time intervals.

The pulse generator 101 features a control switch 1303 for activating the pulse generator 101. In some examples, the pulse generator 101 may feature an electric motor (such as a solenoid motor) coupled to the compression element 1301 to drive the compression element. In other examples, the pulse generator 101 may feature a mechanical/clockwork mechanism that drives the compression element 1301 by means of torsion spring or similar, potentially coupled to a rotatable cam. When the pulse generator 101 uses a clockwork mechanism, the control switch 1303 may be used to wind the clockwork mechanism. An optional second switch (not shown) may also be used to start/stop the clockwork mechanism.

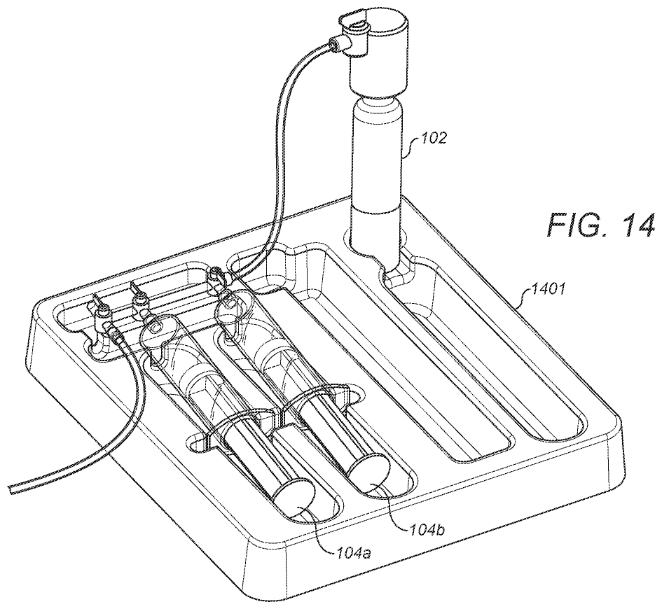

The pulse generator 101 may optionally be supplied in a pre-wound state, for example as part of a kit. Such a kit may additionally contain a packaging element 1401 which acts as a stand for the flushing gas source 102 and the fluid syringes 104a, 104b, as shown in FIG. 14.

While the systems shown in FIGS. 1-7 allow for reliable and effective flushing procedures, correct priming of these systems is important to ensure air is removed from the flushing system before it is used to flush a medical device. The systems described earlier can optionally be used in combination with a priming module that is adapted to separate and capture bubbles that are entrained in a flow of flushing liquid. The priming module may also be referred to as a bubble capture device.

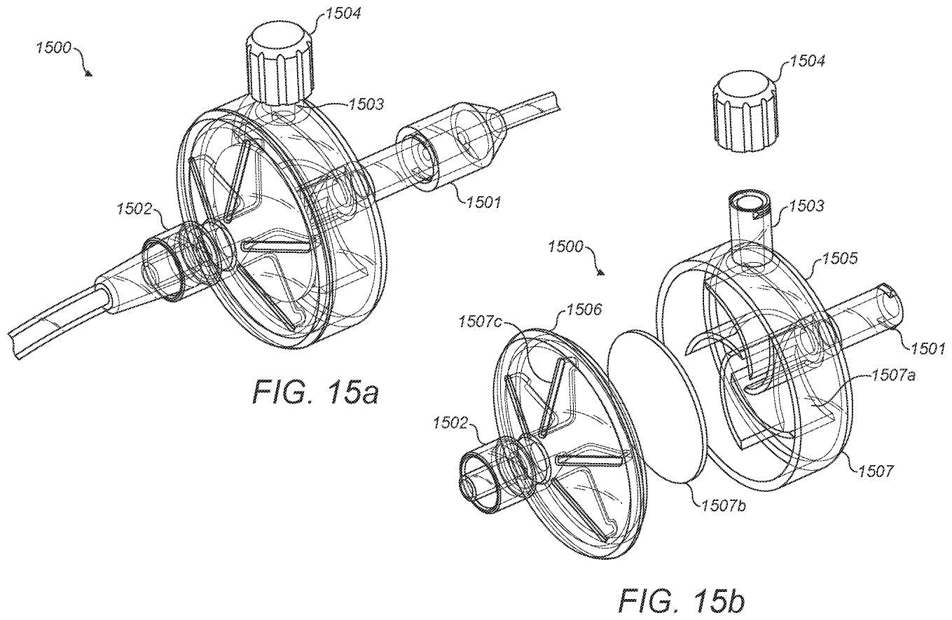

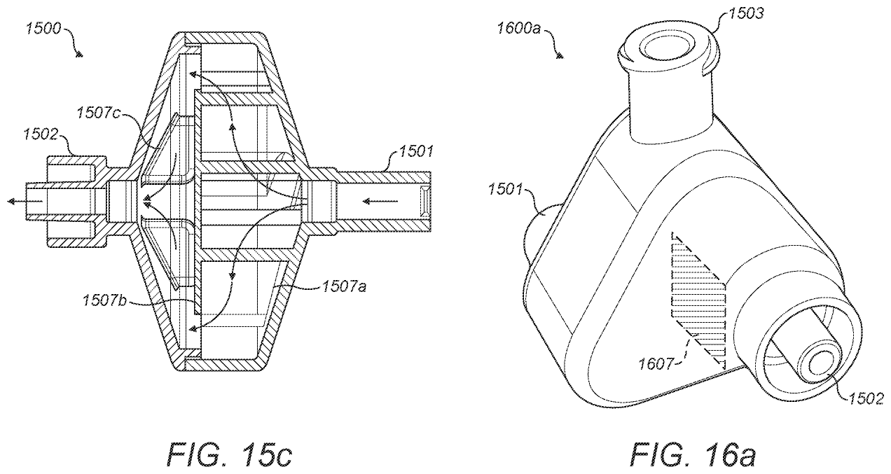

An exemplary priming module 1500 is shown in FIG. 15a. The priming module 1500 is substantially disc shaped (i.e. shaped like a short cylinder) and features an inlet port 1501 and an outlet port 1502 through which a flushing liquid can enter and exit the priming module 1500. The illustrated priming module 1500 also comprises a vent port 1503 with a vent cap 1504 for sealing the vent port 1503. The vent port 1503 may be shaped to couple to a syringe, such as a vacuum pressure syringe. The space inside the priming module 1500 acts as a fluid conduit through which fluids can flow from the inlet port 1501 to the outlet port 1502.

As shown in FIG. 15b, the illustrated priming module 1500 is formed of a first part 1505 and a second part 1506 and further comprises several baffles 1507a-c. The baffles 1507a are formed as curved fins that direct the flushing liquid away from the centre of the first part 1505 in a substantially spiral/helical direction. The baffle 1507b is a disc that prevents flushing liquid flowing directly through the priming module from the inlet port 1501 to the outlet port 1502 and modifies the flow of the flushing liquid to instead flow via the baffles 1507b. The baffles 1507c then act to redirect the flow of flushing liquid radially inward towards the outlet port 1502 of the second part 1506. This flow path is further illustrated in FIG. 15c.

The priming module 1500 may be positioned anywhere in the flushing system before the medical device and after the syringe pump 103, 203. This ensures that all flushing liquids from the fluid syringes 104a, 104b pass through the priming module 1500. Flushing gases can also flow through the priming module 1500, such that it does not need to be removed when flushing with a gas depending on the configuration of the flushing system.

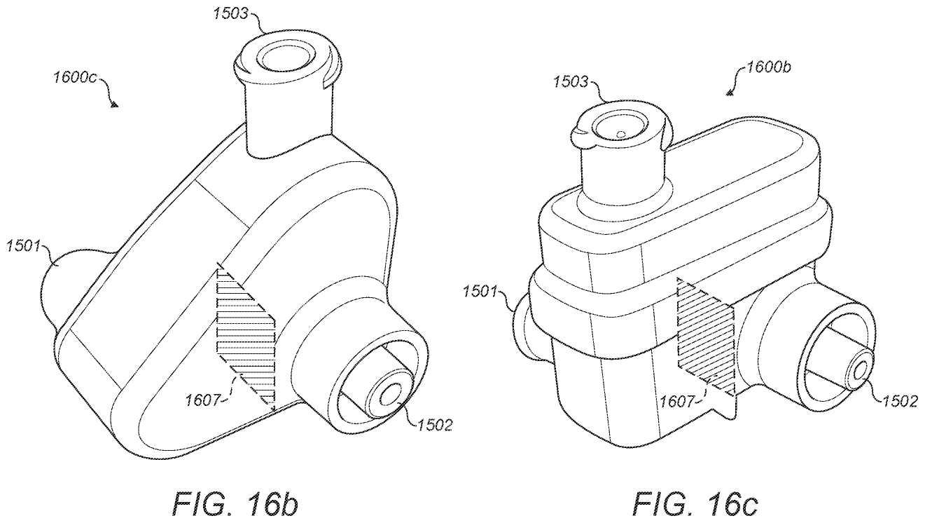

Alternative priming modules 1600a-c are shown in FIGS. 16a-c. Each of these priming modules 1600a-c features an inlet port 1501, outlet port 1502 and a vent port 1503 (which could optionally be provided with a vent cap). Each of the priming modules 1600a-c comprises an internal baffle 1607 positioned to modify the flow of liquid to separate gas bubbles from the liquid; the approximate position of each baffle 1600 is overlaid in FIGS. 16a-c. The space above each baffle forms a headspace in which gas can accumulate.

In addition, in each priming module 1600a-c the outlet port 1502 is offset from the input port 1501 such that flushing liquid cannot flow directly through the priming modules 1600a-c even in the absence of a baffle 1600. The offset nature of the ports adds to the tortuous internal geometry created within the priming modules 1600a-c. In a preferred example, the outlet port 1502 is positioned such that it is lower than the inlet port 1501 when the priming module 1600a-c is in use. This helps to ensure that gases are correctly separated and do not flow through the exit port 1502.

If will be understood that the inlet port 1501 and outlet port 1502 in these examples could alternatively be axially aligned with each other, with a baffle 1607 acting as a weir to disrupt the flow of flushing liquid. In this case, the baffle 1607 would preferably be perpendicular to the axis of alignment (i.e. the line intersecting the inlet port 1501 and outlet port 1502). However, in any of the examples, orientation of the baffle 1607 can be varied provided that the baffle acts to modify the flow of flushing liquid between the inlet port 1501 and the outlet port 1502.

In addition, the priming modules 1600a, 1600b in FIGS. 16a and 16b are shaped to direct or channel separated gas bubbles towards the vent port. In the illustrated examples, this is achieved by having at least one surface that is angled towards the vent port 1503. That is, at least one surface (preferably a top surface) of the priming module 1600a, 1600b is slanted/sloped/inclined towards the vent port 1503, with the vent port 1503 positioned at a topmost point/apex of the priming module 1600a, 1600b.

The internal edges and/or corners of all of the priming modules are preferably curved to prevent gas bubbles accumulating in these regions. However, the baffles may optionally have angled projections (such as angled corners or edges) to assist in agitating the flow of the flushing liquid.

While the priming modules 1600a-c in FIGS. 16a-c each have only one baffle, it will be understood that additional baffles could be used.

The priming modules 1600a-c may be formed from a single piece of material, for example from an elastomeric material such as silicone, or they may be formed of multiple parts that are joined or fused together.

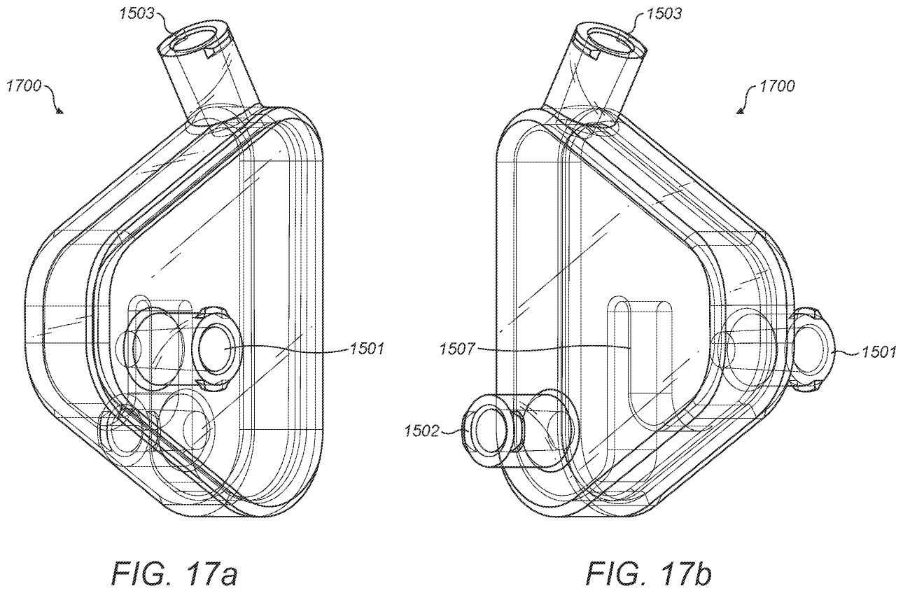

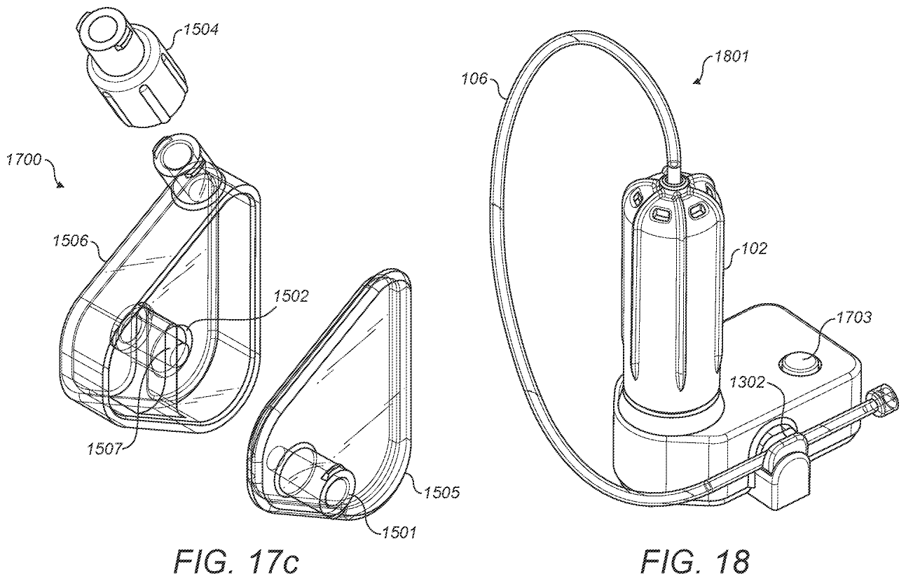

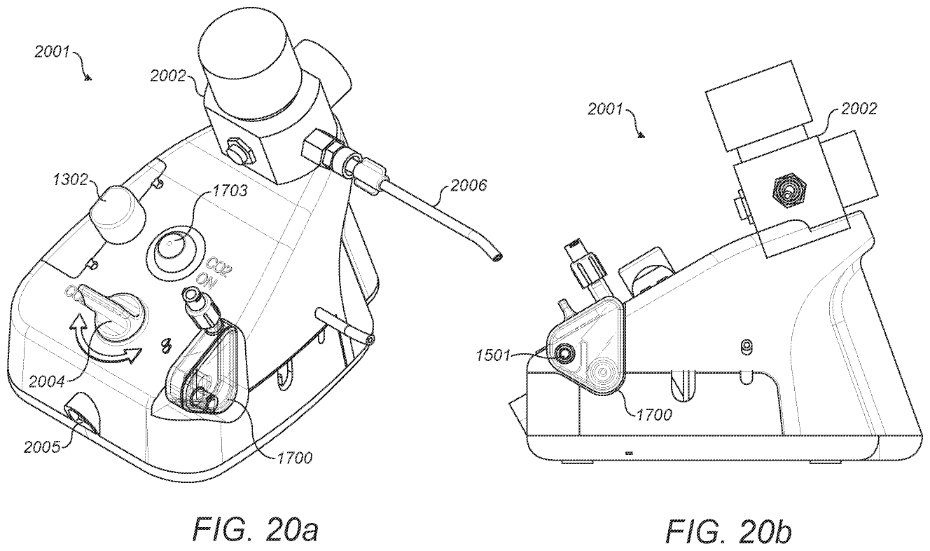

FIGS. 17a-c show another alternative bubble capture device 1700. This bubble capture device 1700 shares the features of the bubble capture devices shown in FIGS. 16a-c described above. The bubble capture device 1700 is shown in the orientation in which it is intended to be used (as also illustrated in FIGS. 20a-b).

As shown in FIGS. 17a-c the outlet port 1502 is lower than the inlet port 1501. As described above, this ensures that gas bubbles are separated and do not flow through the bubble capture device (i.e. the vertically offset ports improve gas separation/capture).

The bubble capture device 1700 is otherwise equivalent to those in FIGS. 16a-c, and all features described in relation to those figures may equally apply to the bubble capture device 1700 in FIGS. 17a-c.

The baffles may also be referred to as deflectors, flow deflectors, flow disruptors, agitators, agitating elements, agitating components or similar, and may be any component that disrupts, agitates or redirects the flow of fluid. The baffles may be ribs, separate components or integrated moulded components for example. The baffles may optionally be formed from an elastomeric material, such as silicone.

The purpose of the baffles is to create a tortuous flow path through the priming module for the flushing liquid, and the baffles of all priming modules disclosed herein are arranged to achieve this purpose. The effect is that the flushing fluid is agitated by the baffles (i.e. laminar flow is disrupted), thereby separating gas bubbles (such as air) from the flow of flushing liquid.

FIG. 18 shows an exemplary pulse generator 1801 that also functions as a stand for the flushing gas source 102 (it could be considered to be a stand that also functions as a pulse generator), and FIG. 18 shows a flushing system 1900 incorporating the pulse generator 1801 and the priming module 1500.

As with the pulse generator 101 illustrated in FIG. 13, the pulse generator 1801 shown in FIG. 18 features a groove or channel 1302 for receiving a connecting tube 106, and the flow of flushing gas through the connecting tube 106 can be restricted by compressing the tube. The pulse generator also features a control switch 1803 for activation.

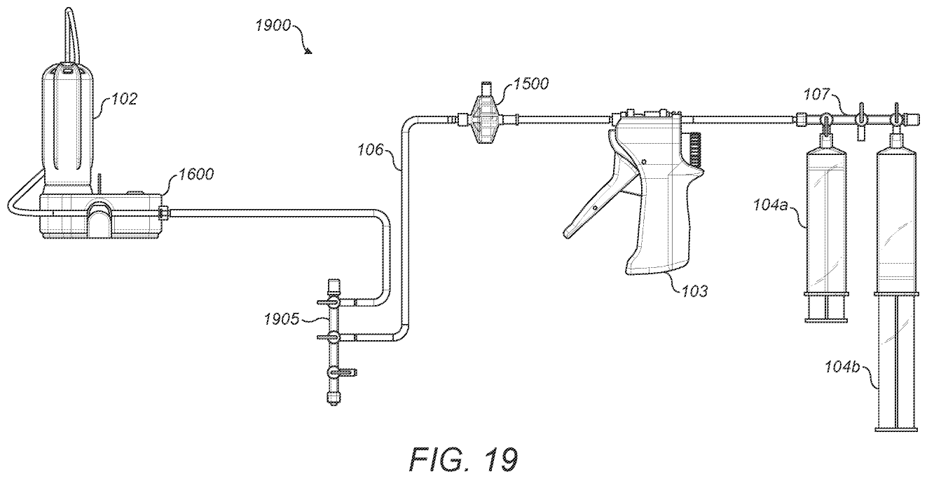

As shown in FIG. 19, the priming module 1500 may be connected directly to a fluid coupling 1905 via a connecting tube 106 such that all flushing liquids from the fluid syringes 104a, 104b pass through the priming module 1500 prior to entering the medical device (which will be attached to the fluid coupling 1905). The illustrated fluid coupling 1905 differs from that in the earlier examples in that it is a multi-valve flushing manifold in to which the flushing gas source 102 and the fluid syringes 104a, 104 are (indirectly) coupled, with the valves being use to selectively couple different flow paths to the medical device (not shown).

FIGS. 20a-b show another exemplary pulse generator 2001 similar to that in FIG. 18. However, the pulse generator 2001 in FIG. 20 is further adapted to receive the pulsed flow of flushing liquid via the bubble capture device 1700 of FIG. 17, which is received in a recess on the side of the pulse generator 2001. As discussed above, the bubble capture device 1700 is oriented such that the outlet port 1502 of the bubble capture device 1700 is lower than the inlet port 1501.

A regulator 2002 is used to control the pressure of the flushing gas (e.g. carbon dioxide) to a predetermined pressure (e.g. 2 bar). The flushing gas then flows through a connecting tube 2006 which loops (not shown) into the body of the pulse generator 2001 and is positioned in the groove or channel 1302 of the pulse generator 2001 as described in the earlier examples.

A flow of pulsed flushing liquid may be coupled to the inlet port 1501 of the bubble capture device 1700. The outlet port 1502 of the bubble capture device 1700 is coupled to a conduit (not shown) within the pulse generator 2001, and a switch 2004 on the pulse generator 2001 is actuatable to select between coupling flows of flushing liquid or flushing gas to an outlet 2005 of the pulse generator 2001. A button 1703 is actuatable to activate and deactivate the pulsed flow of carbon dioxide.

An exemplary method of use of the flushing systems disclosed herein will now be described. It is assumed that all components are connected (including all fluid sources) and that all components have been primed as necessary. It should be understood that this method is intended to be exemplary and should not be construed as limiting. One skilled in the art will appreciate that the steps described below could be performed in a different order whilst still achieving the same ultimate result.

The method preferably begins by flushing the medical device with a flushing gas, such as CO2. The fluid coupling 105, 505, 1905 is controlled to couple the flow of flushing gas from the flushing gas source 102 to the medical device.

The pulse generator 101, 1801 is then activated to repeatedly restrict and allow the flow of flushing gas from the flushing gas source 102 at regular intervals. This activation may involve actuating a control switch 1303, 1803 and/or winding a mechanical clockwork mechanism of the pulse generator 101, 1801. This step can be omitted when a gas trigger 301, 601 is used in place of a pulse generator.

The flushing gas source 102 can then be activated to release flushing gas into the control lines 106, for example by using the valve 1201. If using an automatic pulse generator 101, 1801, the flushing gas source 102 can be activated for a duration sufficient to flush the medical device with the flushing gas (e.g. CO2) and the pulse generator 101, 1801 will automatically (i.e. without further user/operator intervention) cause the flow of flushing gas received at the medical device to be pulsed.

If using a manual gas trigger 301, 601, the operator can actuate the gas trigger 301, 601 at regular intervals to generate the pulsatile flow of flushing gas (as opposed to using an automatic pulse generator 101, 1801.

The purpose of flushing with a flushing gas is to remove as much air as possible prior to flushing with a flushing liquid, with the CO₂ being used to displace the air and replace it with CO2 (which is less harmful than air if released in the body as it can be more readily dissolved in the blood stream). As described earlier, using a pulsed flow of flushing fluid disrupts laminar flow and increases the efficacy of air displacement.

Once the medical device has been sufficiently flushed with the flushing gas, the operator can deactivate the source of flushing gas, for example by using the valve 1201, and the fluid coupling 105, 505, 1905 can be controlled to decouple the flushing gas source 102 from the medical device and to instead couple the syringe pump 103, 203, 603 to the medical device.

The syringe manifold 107 or control knob 801 can be used to select between the fluid syringes 104a, 104b (and to optionally couple them to a vent port 802 or similar for priming prior to flushing the medical device). The trigger component 806 of the syringe pump 103, 203, 603 can then be fully actuated at regular intervals to generate a fixed-volume pulse of flushing fluid from the coupled fluid syringe 104a, 104b. As mentioned earlier, the fluid syringed 104a, 104b preferably contain flushing liquids rather than flushing gases, although the fluid syringe 103, 203, 603 is capable of delivering pulses of both gases and liquids (provided any flushing gas in the syringes 104a, 104b is in an uncompressed/unpressurised state). Pulses of the flushing fluid can then be delivered as necessary to sufficiently flush the medical device, with a pulse being generated each time the trigger component 806 is actuated. The procedure can then be repeated with flushing fluid from the other fluid syringe 104a, 104b if needed, and additional fluid syringes could also be used.

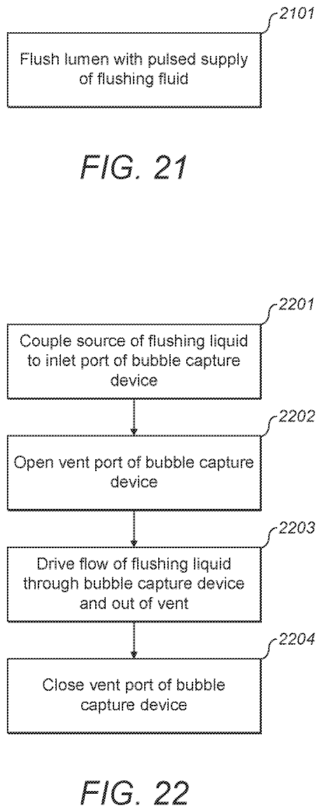

FIG. 21 illustrates a flushing method that can be performed using the systems described earlier.

At step 2101, a lumen of a medical device is flushed with a pulsed supply of flushing fluid. The lumen may optionally contain a further medical device, such as a stent, which is also to be flushed. The method may additionally involve coupling the lumen to the pulsed supply of flushing fluid.

The flushing fluid may be a flushing gas, such as carbon dioxide, or a flushing liquid, such as perfluorocarbon or a saline solution, which may optionally be a buffer solution, degassed and/or pH-adjusted. A degassed solution will absorb quantities of gases (such as carbon dioxide or air) that it comes into contact with, thereby enhancing removal of gases from the medical device.

The method may involve a first flushing step with a pulsed supply of a flushing gas and a subsequent flushing step with a pulsed supply of a flushing fluid. In this way, the gas can be used to displace air from the medical device, and the flushing liquid can be used to displace and/or absorb the flushing gas and residual air, for example pockets of air that are trapped in the medical device.

In some examples, the method may involve flushing the lumen at a pressure above 101.325 kPa, which is standard pressure. Flushing at higher pressures increases the flushing fluid's ability to absorb air.

Prior to flushing the medical device, the priming module 1500, 1600a-c may be used to assist in removing air from the flushing system. In an exemplary method, fluid (such as saline solution) may be pumped through components of the system via the priming module 1500, 1600a-c with the vent port 1503 open to allow air to escape. Additionally or alternatively, a negative pressure source, for example a vacuum pressure syringe such as a VacLok® syringe, may be connected to the vent port 1503 and used to draw a flushing fluid through the components of the system.

FIG. 22 illustrates such a method for priming a catheter flushing system, for example one of systems in FIGS. 1-7 or FIG. 19.

At step 2201, a source of flushing liquid is coupled to an inlet port of a bubble capture device (e.g. one of the priming modules shown in FIGS. 15 and 16).

At step 2202, an air vent or vent port of the bubble capture device is opened.

At step 2203, a flow of the flushing liquid is driven through the bubble capture device and out of the vent port.

At step 2204, the vent port is closed.

The flow of flushing liquid could be driven by a pump coupled to the source of flushing liquid, or it could alternatively be driven by a vacuum source coupled to the vent port, such as a vacuum pressure syringe.

In all of the above examples, any appropriate pulsing frequency may be used. Particularly effective frequencies include pulsing frequencies between five times per second and once every 10 seconds inclusive (0.1 Hz to 5 Hz inclusive), preferably between two times per second and once every five seconds inclusive (0.2 Hz to 2 Hz inclusive), even more preferably between two times per second and once every two seconds inclusive (0.5 Hz to 2 Hz). Different pulsing frequencies may optionally be used for different flushing fluids (e.g. a different frequency may be used when flushing with gases to that used when flushing with liquids).

As explained earlier, various components in the systems and devices described above could be combine and/or swapped, and the above examples are not intended to be limiting. Similarly, unless indicated otherwise, the order of method steps described above is intended to be exemplary rather than limiting, and one skilled in the art will appreciate that the order of certain method steps could be changed without affecting the end result.

Although the priming module described above is preferably used in combination with one of the systems described above, it is also suitable for use with other flushing systems, such as those that use flushing liquids only (i.e. not flushing gases).

Likewise, although the pulsed flushing method disclosed herein is preferably used in combination with the one of the systems described above, it may alternatively be used with any other suitable flushing system.

In addition, although the method is preferably used to flush a lumen of a medical device such as catheter, the systems and methods disclosed herein could be used to flush any suitable medical device, in particular when disposed within a lumen. For example, a stent graft of similar may be flushed while within a catheter lumen. The methods may also be used to flush medical devices disposed in other receptacles (i.e. they are not only suitable for flushing lumens of medical devices and medical devices disposed therein).

1. A system for flushing a lumen of a medical device to remove air, comprising:
a first fluid delivery device adapted to provide a pulsatile flow of a flushing gas from a pressurised source of the flushing gas;
a second fluid delivery device adapted to provide a pulsatile flow of a flushing liquid from a source of the flushing liquid; and,
at least one fluid coupling for connecting the first fluid delivery device and the second fluid delivery device to the lumen of the medical device, wherein the first fluid delivery device comprises a flow restrictor that is actuatable to restrict a flow of the flushing gas to thereby pulse the flow of the flushing gas.

2. The system of claim 1, wherein the restrictor is actuatable between an open position and a closed position to thereby pulse a flow of the flushing gas.

3. The system of claim 1, wherein the first fluid delivery device comprises a compression element configured to compress a flexible tube coupled to the source of the flushing gas at predetermined time intervals to thereby restrict a flow of the flushing gas through the tube.

4. The system of claim 3, wherein the compression element is reciprocally movable.

5. The system of claim 4, wherein the compression element comprises a solenoid motor.

6. The system of claim 3, wherein the compression element comprises a rotatable cam.

7. The system of claim 6, wherein the rotatable cam is coupled to a torsion spring adapted to drive the rotatable cam.

8. The system of claim 3, wherein the compression element is coupled to an electric motor configured to drive the compression element.

9. The system claim 3, wherein the compression element is pneumatically drivable, preferably wherein the compression element is pneumatically drivable by the flushing gas.

10. The system of claim 1, wherein the first fluid delivery device comprises a user actuatable gas control mechanism for providing the pulsatile flow of the flushing gas.

11. The system of claim 1, further comprising a stand adapted to retain a source of the flushing gas in an upright position, preferably wherein the stand comprises the first fluid delivery device.

12. The system of claim 1, wherein the second fluid delivery device comprises a user actuatable liquid delivery mechanism for providing tea pulsatile flow of the flushing liquid, preferably wherein the user actuatable liquid delivery mechanism is a trigger.

13. The system of claim 12, wherein the second fluid delivery device comprises a user actuatable positive displacement pump.

14. The system of claim 13, wherein the positive displacement pump comprises a compression chamber.

15. The system of claim 14, wherein the positive displacement pump comprises a one-way valve configured to allow a one-way flow of flushing liquid from the source of the flushing liquid into the compression chamber.

16. The system of claim 13, wherein the positive displacement pump comprises a one-way valve configured to allow a one-way flow of flushing liquid from the positive displacement pump towards the at least one fluid coupling.

17. The system of claim 13, wherein the positive displacement pump comprises one or more resilient compression members arranged to reset the user actuatable liquid delivery mechanism to an initial position and/or draw the flushing liquid from the source of flushing liquid fluid source.

18. The system of claim 1, wherein the at least one fluid coupling is a three-way valve.

19. The system of claim 1, wherein the first and second fluid delivery devices are comprised in a single device.

20. The system of claim 1 wherein the first fluid delivery device is couplable to the at least one fluid coupling via the second fluid delivery device.

21. The system of claim 1, wherein the flushing liquid is a first flushing liquid, and wherein the second fluid delivery device is further adapted to provide a pulsatile flow of a second flushing liquid from a source of the second flushing liquid, preferably wherein the second fluid delivery device comprises control means for selectively coupling the second fluid delivery device to the respective sources of the first and the second flushing liquids.

22. The system of claim 1, further comprising a sterile filter positionable in-line between the first fluid delivery device and the pressurised source of the flushing gas.

23. A method for flushing a lumen of a medical device to remove air prior to introducing the medical device within a body, comprising:
flushing the lumen with a pulsed supply of a flushing gas from a pressurised source of flushing gas via a first fluid delivery device, and with a supply of flushing liquid from a source of the flushing liquid via a second fluid delivery device, and
actuating a flow restrictor to restrict the flow of the flushing gas of the first fluid delivery device.

24. (canceled)

25. (canceled)

26. The method of claim 23, wherein the flushing gas is carbon dioxide.

27. (canceled)

28. The method of claim 23, wherein the flushing liquid is a buffer solution.

29. The method of claim 23, wherein the flushing liquid is pH adjusted

30. The method of claim 23, wherein the flushing liquid comprises saline.

31. The method of claim 23, wherein the flushing liquid is degassed.

32. The method of claim 23, wherein the flushing liquid is a perfluorocarbon solution.

33. The method of claim 23, wherein flushing the lumen comprises flushing at a pressure above 101.325 kPa.

34. The method of claim 23, wherein flushing the lumen comprises coupling the lumen to the pulsed supply of flushing fluid.

35.-50. (canceled)

51. The system of claim 1, wherein the second fluid delivery device is adapted to provide a pulsatile flow of flushing fluid.
